(19) United States
(12) Patent Application Publication
(10) Pub. No.: US 2021/0212756 A1
(43) Pub. Date: Jul. 15, 2021

(54) TISSUE CUTTING SYSTEMS AND METHODS

(71) Applicant: Nasser Rafiee, Andover, MA (US)

(72) Inventor: Nasser Rafiee, Andover, MA (US)

(21) Appl. No.: 17/148,616

(22) Filed: Jan. 14, 2021

Related U.S. Application Data
(63) Continuation-in-part of application No. 16/563,925, filed on Sep. 8, 2019.
Application No. 16/563,925 is a continuation-in-part of application No. PCT/US2018/048177, filed on Aug. 27, 2018.
Application No. PCT/US2018/048177 is a continuation-in-part of application No. PCT/US2020/055160, filed on Oct. 9, 2020.
(60) Provisional application No. 62/550,347, filed on Aug. 25, 2017.
(60) Provisional application No. 62/567,203, filed on Oct. 2, 2017.
(60) Provisional application No. 62/663,518, filed on Apr. 27, 2018.
(60) Provisional application No. 62/688,378, filed on Jun. 21, 2018.
(60) Provisional application No. 62/712,194, filed on Jul. 30, 2018.
(60) Provisional application No. 62/728,413, filed on Sep. 7, 2018.
(60) Provisional application No. 63/047,995, filed on Jul. 3, 2020.
(60) Provisional application No. 63/077,579, filed on Sep. 12, 2020.

(51) Int. Cl. A61B 18/14; A61B 18/12; A61B 90/00

(52) U.S. Cl. CPC A61B 18/1492; A61B 18/1206; A61B 2018/00601; A61B 2018/147; A61B 90/39

(57) ABSTRACT

The disclosure provides various embodiments of catheters having articulable ends that can be used for various procedures. Embodiments of methods are also provided that can be performed with catheters in accordance with the present disclosure.

### CROSS-REFERENCE TO RELATED APPLICATION

The present patent application is a continuation-in-part of and claims the benefit of priority to International Application No. PCT/US20/55160, filed Oct. 9, 2020. The present patent application also claims the benefit of priority to U.S. Patent Application No. 63/047,995, filed Jul. 3, 2020 and U.S. Patent Application No. 63/077,579, filed Sep. 12, 2020. The present patent application also claims the benefit of priority to and is a continuation-in-part of U.S. patent application Ser. No. 16/563,925, filed Sep. 8, 2019, which in turn claims the benefit of U.S. Patent Application Ser. No. 62/728,413, filed Sep. 7, 2018, and International Patent Application No. PCT/US18/48177, filed Aug. 27, 2018, which in turn claims the benefit of priority to U.S. Provisional Application Ser. No. 62/550,347, filed Aug. 25, 2017, U.S. Provisional Application Ser. No. 62/567,203, filed Oct. 2, 2017, U.S. Provisional Patent Application Ser. No. 62/663,518, filed Apr. 27, 2018, U.S. Provisional Application Ser. No. 62/688,378, filed Jun. 21, 2018, and U.S. Provisional Patent Application Ser. No. 62/712,194, filed Jul. 30, 2018.

### BACKGROUND

The disclosure relates generally to medical treatment devices and techniques, and, in some aspects, to methods and devices for diagnosis and treatment of cardiac valves. The present disclosure provides improvements over the state of the art.

### SUMMARY OF THE DISCLOSURE

The present disclosure provides various systems and methods for removing clips, cysts and other structures from valve leaflets. The disclosure further provides systems for modifying or removing luminal valve leaflets. The disclosure also provides other innovations, as set forth below.

### BRIEF DESCRIPTION OF THE FIGURES

FIGS. 1A-2B are illustrations of a first device in accordance with the present disclosure.

FIGS. 3A-3C show aspects of a first embodiment of a guide wire used in an electrosurgical procedure in accordance with the disclosure.

FIG. 4 shows aspects of a second embodiment of a guide wire used in an electrosurgical procedure in accordance with the disclosure.

FIGS. 5A-5B present views of a grasping catheter or manipulator in accordance with the present disclosure.

FIGS. 6-9 illustrate aspects of a guidewire in accordance with the present disclosure.

FIGS. 10A-12B illustrate aspects of a further guidewire denuding system in accordance with the disclosure.

FIGS. 13-22 illustrate aspects of still a further guidewire denuding system in accordance with the disclosure.

### DETAILED DESCRIPTION

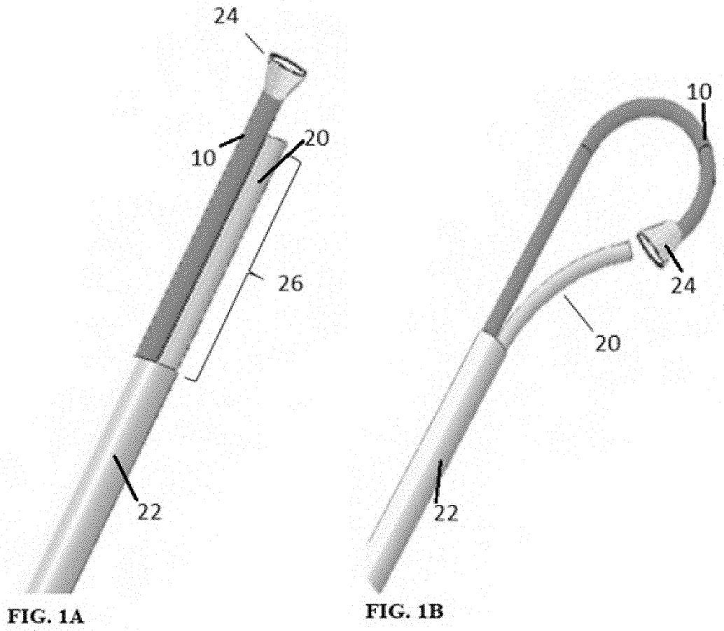

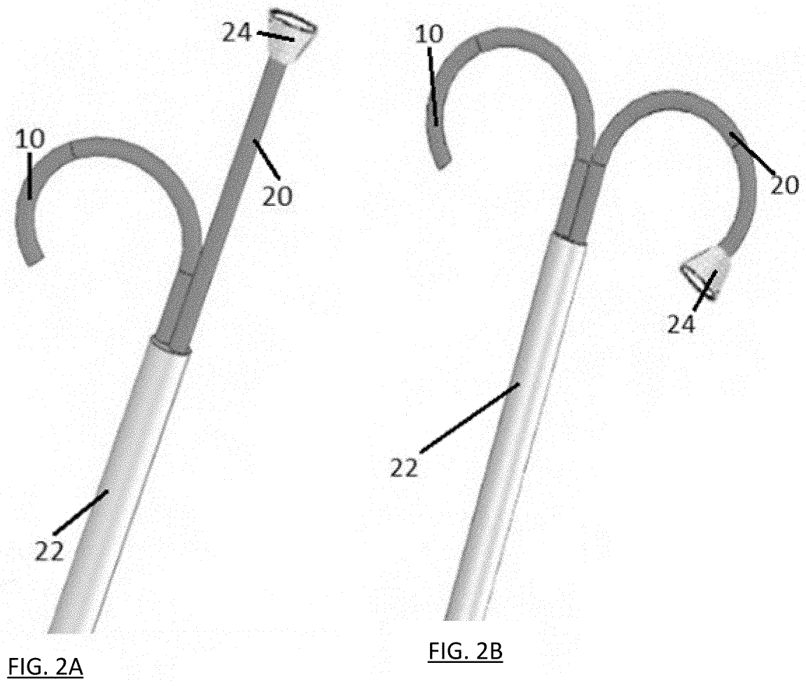

For purposes of illustration, and not limitation, exemplary embodiments of a catheter, which can also be used as a robotic manipulator, are presented in FIGS. 1A-2B. For purposes of simplicity but not limitation, the devices are typically referred to herein as “catheters” but it will be understood by those of skill in the art that they can equally be considered to be robotic manipulators. Thus, all embodiments herein can be provided with manual actuators at their proximal end as is the case with catheters typically, or they can instead be connected to a robotic or pantograph manipulator system including but not limited to those manufactured by Intuitive Surgical of Sunnyvale, Calif. Such robotic or remote actuators can be found, for example, in U.S. patent application Ser. No. 15/580,629, filed Dec. 7, 2017 which is incorporated by reference herein in its entirety for all purposes.

With reference to FIGS. 1A-2B, an elongate catheter is provided having a proximal end and a distal end. The catheter includes an elongate tubular main body 22 having a proximal end, a distal end, and defining at least one elongate passage therethrough. The elongate tubular main body defining a longitudinal axis along its length.

The catheter includes a first elongate inner body 10 having a proximal end and a distal end. The inner body 10 is illustrated with an illustrative cone-shaped atraumatic distal tip 24 that is configured to spread applied stress out over a larger area, which can be of particular benefit when contacting delicate anatomical structures. The first elongate inner body 10 is slidably disposed within the at least one elongate passage of the elongate tubular main body 22.

Also illustrated is a second elongate inner body 20 having a proximal end and a distal end that is slidably disposed within the at least one elongate passage of the elongate tubular main body 22, which is suitably configured to maintain registration of bodies 10 and 20 with respect to each other and hold them together. Bodies 10 and 20 can be housed in a common passage, or in individual passages defined within body 22. Body 20 is slidably disposed with respect to the first inner body 10, wherein an exposed distal region 26 of body 20 is illustrated as protruding beyond the distal end of main body 22.

As illustrated, the distal end of the first and second inner elongate bodies 10, 20 are preferably biased or otherwise configured to be curled or steered away from the longitudinal axis in a proximal direction when the first elongate inner body is advanced distally with respect to the main body by virtue of inner body 10 being removed from body 22. Bodies 10, 20 can be configured to curl as illustrated when advanced distally from body 22 by making bodies 10, 20 at least in part from shape memory materials, and/or by utilizing a steering wire that travels the length of the body 10, 20 that is attached to a distal end of each of the bodies 10, 20, such as by way of a ring (e.g., a radiopaque marker band) that is attached to the distal end of the bodies. In another embodiment, one or more of bodies 10, 20 can be formed at least in part by thermoplastic or other polymeric or composite material that is molded with a preformed bend therein. Such a pre-bent or pre-formed body 10, 20 can then be loaded, for example, into main body 22, wherein main body 22 maintains the bodies 10, 20 in a straight orientation until they bodies 10, 20 are advanced distally with respect to main body 22, at which time they revert to their curved shape and regain at least some of their original curvature.

Main body 22 can simply be an overwrap or a sheath in some implementations that functions to maintain the bodies 10, 20 in a parallel relationship and optionally maintains the bodies 10, 20 in a relative orientation until the bodies 10, 20 are advanced distally with respect to body 22. In other implementations, body 22 can be more sophisticated such as a multi-lumen extrusion including a plurality of lumens for slidably containing bodies 10, 20, and other devices, as desired. In lieu of a main body 22 or overwrap, bodies 10, 20 can alternatively be fused or adhered to each other, or be provided with an adjustable coupling that runs their lengths that permits relative slidability of bodies 10, 20.

If desired, each of the first elongate inner body 10 and second elongate inner body 20 can each define one or more lumens along their respective lengths. The lumen(s) can be used, for example, for passage of a further medical instrument such as a guidewire or viewing scope, for directing electrical conductors, and the like, and/or for passage of a steering wire along the length of body 10, 20 terminating, for example, in a marker band at the distal end of body 10, 20 that the steering wire attaches to. Other examples of suitable steering mechanisms can be found in U.S. Pat. Nos. 6,030,360, and 6,579,278, which are incorporated by reference herein in their entireties for any purpose whatsoever. Either body 10, 20 if equipped with such a passage can additionally or alternatively include a movable body (e.g., core wire, snare catheter, etc.) slidably disposed therein.

If the passage within body 10 includes a snare catheter (such as that described in U.S. patent application Ser. No. 13/824,198, filed May 1, 2013), the snare catheter can be directed out of the distal end of body 10 to provide a landing or target zone for a guidewire that is directed through the distal end of body 20 (not shown). This permits a guidewire that traverses through the distal end of the body 20 to be captured by the snare catheter that extends outwardly from body 10, thereby permitting the guidewire extending from the distal end of body 20 to be pulled into the distal end of body 10, and advanced through the body 10 and externalized or otherwise directed out of the proximal end of body 10 (not shown).

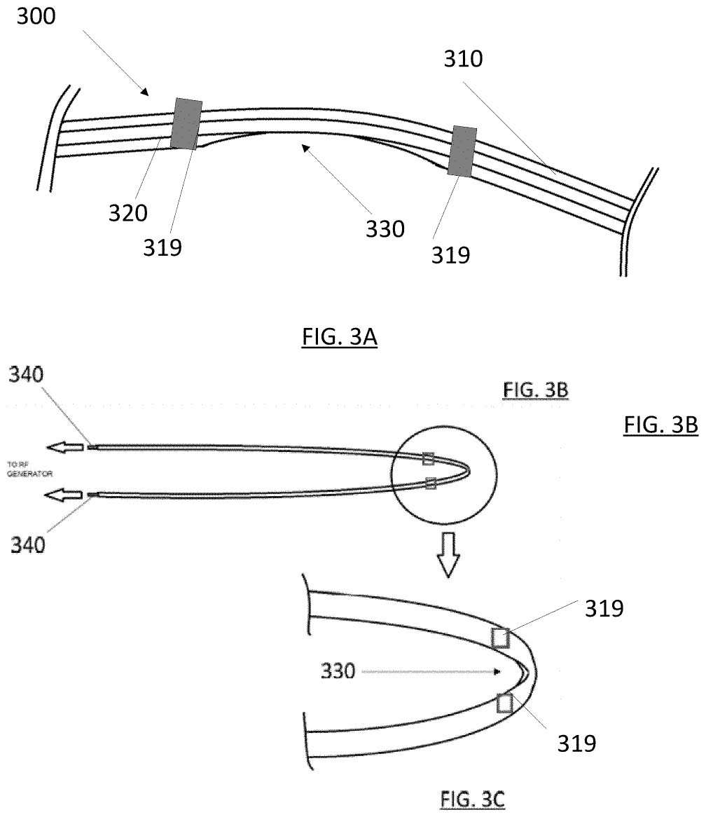

If desired, the guidewire disposed in body 20 can include an electrically conductive core wire surrounded by a jacket made from dielectric/insulating material. The jacket can be removed from a portion of the core wire to expose a portion of the core wire. In a further embodiment, as illustrated in FIGS. 3A-3C, the guidewire can include a core wire that is in turn surrounded by a first insulating layer. As illustrated in FIG. 3A, the guidewire 300 can have an electrically conductive core wire 320 surrounded by a jacket 320 made from dielectric material, such as PTFE or other suitable material. The jacket can be stripped off on one side to create an exposed region 330 of the core wire 320. The exposed region can include one or more marker bands 319 to denote either end of the exposed region 330, or may be placed in any other desired location to enhance visibility of the exposed region 330 under visualization in actual use. The ends of the core wire 320 can likewise be exposed, and the wire can be bent in half so that the exposed core wire 320 faces itself. When the exposed ends 340 of the core wire 320 are then connected to a generator (not shown) in a bipolar arrangement in this case to cause current to pass through the core wire, in the exposed region of the core wire that is bent over, an electrical discharge, or arc, can develop that jumps across the gap (rather than the current passing only along the core wire) that can be used to help cut and/or burn through tissue by pulling the exposed wire through the tissue.

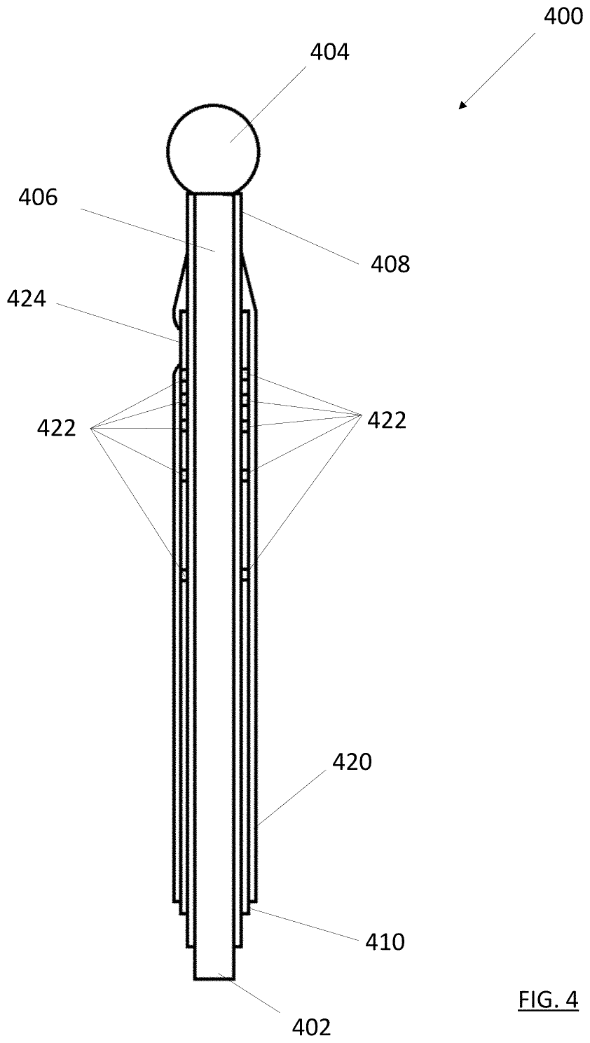

If desired, the guidewire can be provided with more than one conducting layer as embodiment 400 in FIG. 4. Guidewire 400 has an exposed proximal end 402 connected to a distal tip (in this case in the shape of a metallic ball 404, and an elongate core wire 406. A first insulating layer 408 (made of a polymeric layer, for example), is disposed about the core wire 406 along its length, but leaving the tip 404 and proximal end 402 exposed. Proximal end 402 can be electrically coupled to a signal generator, and the current can pass, for example, through the distal tip 404 and follow a return path to a conductive path (not shown) through the patient's body (monopolar arrangement) to the electrical generator. This is a useful arrangement for cutting through tissue with the tip of the guidewire 400. If desired, a beneficial agent may be injected through one or more arms of the system to the cutting site, such as nonionic 5% dextrose, in order to reduce the non-target conduction and enhance to laceration. Guidewire 400 further includes a second electrical conductor, or conducting layer, 410, is disposed at least partially about, or at least radially outwardly from, the first insulating layer 408. The second electrical conductor/conducting layer 412, in turn, can in turn be surrounded by an outer insulating layer 420. The outer insulating layer can be removed to expose a portion of the second electrical conductor/conducting layer to define an exposed portion 424 of the second electrical conductor/conducting layer. As illustrated, portion 424 is facing laterally outwardly to permit a cut to be performed by moving the guidewire 400 laterally to the side, when a proximal end of the layer 410 is attached to a signal generator. Current then flows through the exposed portion 424 and through the tissue to a conductive pad that is attached to a return path of the signal generator. Conductive layer 410 can be a continuous layer, such as a tubular layer, or can be an interrupted layer, wherein a conductive path is nonetheless maintained from the exposed patch 424 to the proximal end of the conductive layer 410.

Conductive layer 410 can be formed, for example, from a metallic tube, such as a hypotube, in turn be defined by a tubular body that defines at least one opening 422 therethrough. For example, the at least one opening can be spiral shaped (via laser cutting) and winds around the first insulating layer, resulting in the remaining conductive material also winding around the first insulating layer. Alternatively, the at least one opening and the tubular body define a plurality of articulating segments, similar to those defined in U.S. Pat. No. 8,530,783, Feb. 3, 2010, U.S. Pat. No. 5,605,543, filed Jan. 30, 1996, U.S. patent application Ser. No. 10/969,088, filed Oct. 20, 2004, or WO2017117092, each of which is incorporated by reference herein in its entirety for any purpose whatsoever.

The disclosure also provides an electrosurgical system including a radio frequency power supply, such as that described in U.S. Pat. No. 6,296,636, which is incorporated by reference herein in its entirety for any purpose whatsoever operably coupled to the electrically conductive core wire of the elongate catheters (and/or of the second conductors of catheters) disclosed herein. Thus, the radio frequency power supply can be operably (and selectively) coupled to the electrically conductive core wire and to the second electrical conductor, as desired. Similarly, the disclosure also provides an ultrasonic surgical system, such as an ultrasonic scalpel, including an ultrasonic power source, such as that disclosed in U.S. Pat. No. 6,514,267, which is incorporated by reference herein in its entirety for any purpose whatsoever.

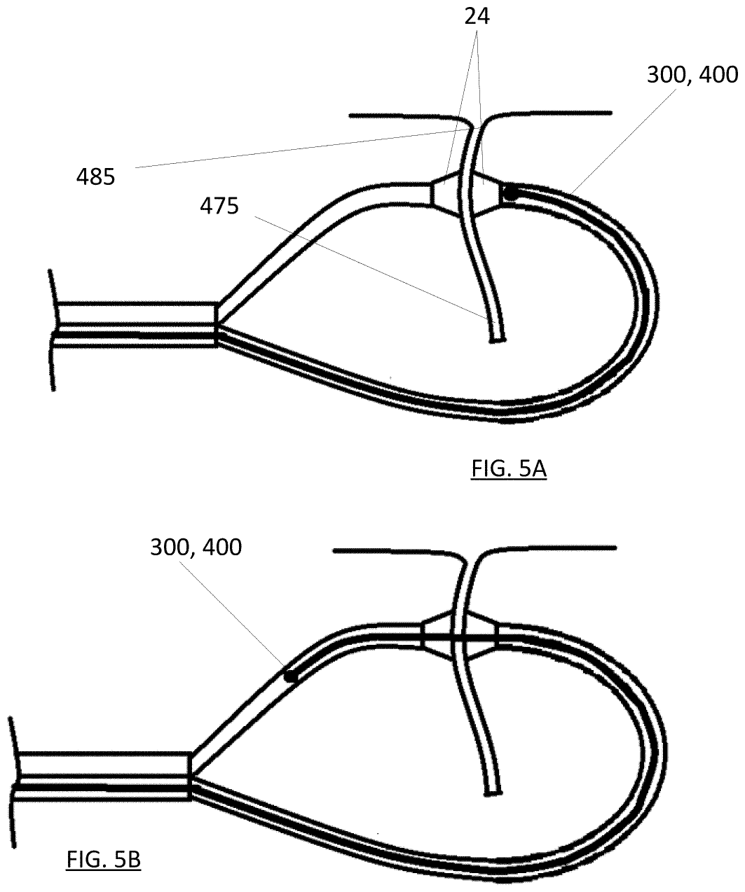

In further embodiments, and with reference to FIGS. 5A-5B, the body (e.g., 20) of the catheter can be configured so as to penetrate an anatomical structure, such as a heart valve leaflet 475, prior to passing into the lumen of the first elongate inner body. Tip(s) 24 of the catheter can grip the leaflet and align the passages in the arms of the catheter to permit a guidewire (e.g., 300, 400) to pierce the leaflet and pass through the catheter arms. Piercing can be accomplished (preferably under imaging, such as fluoroscopy) with a sharpened tip and cuff connection, electrosurgical or ultrasonic cutting tip (e.g., 404). Typically, the leaflet (e.g., 475) is penetrated or pierced in a region that is near or in the annulus 485 of the valve leaflet, most preferably where the annulus transitions to the leaflet base.

The disclosed embodiments of articulating catheters can be used to perform the procedures described in the journal publications annexed to U.S. Provisional Application Ser. No. 62/567,203, filed Oct. 2, 2017. These articles are set forth in the Appendix of that Application and include (i) “Intentional Percutaneous Laceration of the Anterior Mitral Leaflet to Prevent Outflow Obstruction During Transcatheter Mitral Valve Replacement”, Babaliaros et al, J.A.C.C.: Cardiovascular Interventions, Vol. 10, No. 8, 2017, and (ii) “Intentional Laceration of the Anterior Mitral Valve Leaflet to Prevent Left Ventricular Outflow Tract Obstruction During Transcatheter Mitral Valve Replacement”, J.A.C.C.: Cardiovascular Interventions, Vol. 9, No. 7, 2016. The portion of 62/567,203 including the aforementioned publications is hereby incorporated by reference in its entirety. When an electrically exposed portion of the guide wire is in alignment with the leaflet, the ends of the catheter can be withdrawn partially, the electrical current can be turned on, and the exposed portion of the guidewire can be pulled through the leaflet, cutting the leaflet.

Any suitable power level and duty cycle can be used in accordance with the disclosed embodiments. For example, continuous duty cycle (cutting) radiofrequency (“RF”) energy can be used, for example, at a power level between about 50 and 100 Watts, or any increment therebetween of about one watt. The cuts can be made by applying power for between about one half of a second and about five seconds, or any increment therebetween of about one tenth of a second.

As a further example, the movable body (e.g., 20, or a slidable device within a lumen defined by body 20) can include a dart passer that is configured to advance a dart having a suture attached thereto out of the distal end of the second elongate inner body and into a receiving cuff disposed in the lumen of the first elongate inner body, in accordance with the teachings of US2013/0310853, which is incorporated by reference herein in its entirety for any purpose whatsoever. For example, the receiving cuff can be disposed within a lumen defined in body 10 at is attached to a filament/suture that passes through the lumen of body 10 that can receive a dart attached to or resting on the distal end of a hypotube that is advanced through body 20, wherein the dart has a trailing suture that passes through the body of the hypotube. After connecting the dart and cuff, the suture attached to the cuff or the suture attached to the dart can be advanced withdrawing the coupling from the patient, and leaving behind the looped suture.

In accordance with further aspects, the rotational position of the first elongate inner body 10 can be fixed with respect to the rotational position of the second elongate inner body 20. Or, if desired, the rotational positions of each of body 10 and 20 can be controlled by a user at a control actuator/Luer lock at a proximal location of the catheter.

Each of bodies 10, 20 can be made from a variety of materials, including multilayer polymeric extrusions, such as those described in U.S. Pat. No. 6,464,683 to Samuelson or U.S. Pat. No. 5,538,510 to Fontirroche, the disclosure of each being incorporated by reference herein in its entirety. Other structures are also possible, including single or multilayer tubes reinforced by braiding, such as metallic braiding material. Any of the catheters, manipulators, guidewires, or other catheters disclosed herein or portions thereof (e.g., portions 10, 20) can be provided with regions of varying or stepped-down stiffness with length using any of the techniques set forth in U.S. Pat. No. 7,785,318, which is incorporated by reference herein in its entirety for any purpose whatsoever.

Any surface of various components of the system described herein or portions thereof can be provided with one or more suitable lubricious coatings to facilitate procedures by reduction of frictional forces. Such coatings can include, for example, hydrophobic materials such as PolyTetraFluoroEthylene (“PTFE”) or silicone oil, or hydrophilic coatings such as Polyvinyl Pyrrolidone (“PVP”). Other coatings are also possible, including, echogenic materials, radiopaque materials and hydrogels, for example.

One or more actuators can be provided to actuate relative proximal and distal movement of bodies 10, 20 with respect to main body 22. Such actuators typically provide either two handles for push-pull actuation, or the actuator can be more exotic. For example, it is also possible to use other actuators as are known in the art, such as threaded rotating actuators similar to those for retractable sheaths as described in U.S. Pat. No. 6,488,694 to Lau and U.S. Pat. No. 5,906,619 to Olson, the specifications of which are incorporated herein by reference.

With reference to FIGS. 3-4, the disclosure also provides a method that includes providing an electrosurgical system as described hereinabove, deploying the distal end of the catheter into a patient's vasculature to a target location proximate the patient's valve, deploying the first elongate inner body so that the distal end of the first elongate inner body curls around the edge of the patient's valve leaflet, deploying the second elongate inner body so that the distal end of the second elongate inner body bends toward the distal end of the first elongate inner body, directing the guidewire out of the distal end of the second elongate inner body, through the patient's valve leaflet near the valve annulus (such as where the annulus transitions to the base of the leaflet), and into the lumen of the first elongate inner body, advancing the guidewire until the exposed portion of the core wire or second conductor is located in a gap defined between the distal end of the first elongate inner body and the distal end of the second elongate inner body that coincides with the valve leaflet, wherein the exposed portion of the core wire is facing in a proximal direction, energizing the power supply of the electrosurgical system, and advancing the exposed portion of the core wire or second conductor through at least a portion of the valve leaflet to effectuate a cut in the valve leaflet.

As described herein, when practicing the illustrative methods, the exposed portion of the core wire or second conductor can be advanced through the valve leaflet through a peripheral edge of the valve leaflet. In some implementations, the valve leaflet can be a mitral valve leaflet, such as a native or artificial/replacement anterior or posterior mitral valve leaflet, or a native or artificial/replacement tricuspid, pulmonary or aortic valve leaflet. It will be appreciated that the disclosed systems can be used with respect to any suitable native or artificial/replacement valve leaflet.

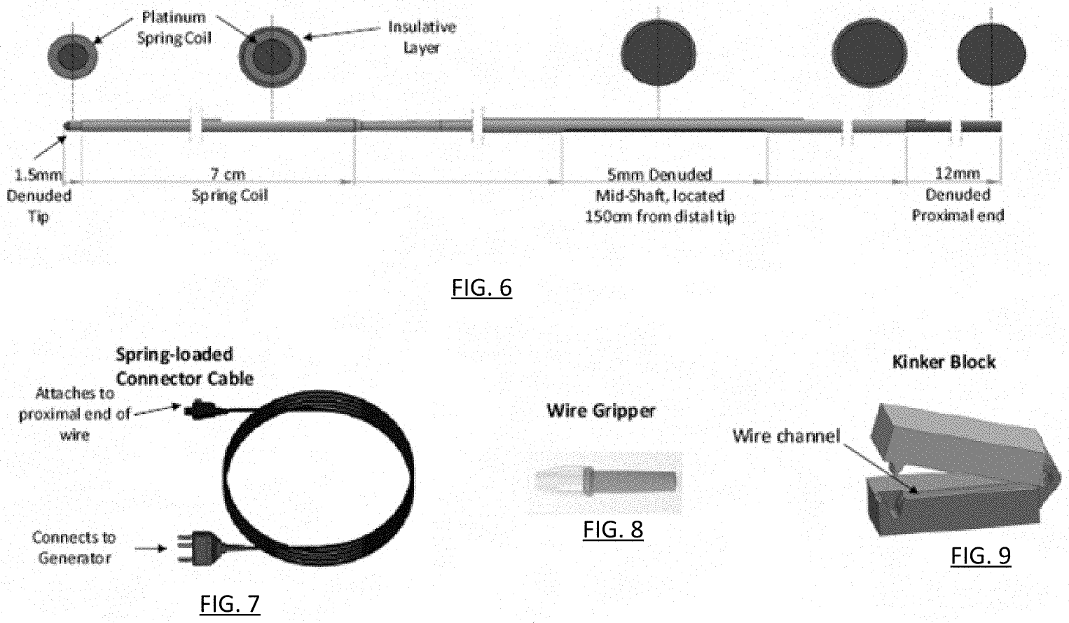

FIGS. 6-9 illustrate an example of a guidewire and the use thereof to carry out procedures also described elsewhere in the present application. The depicted guidewire is useful to cut soft tissue. It can be used under fluoroscopic guidance during procedures where tissue cutting or traversal is required. The guidewire typically has two elements that can interact with tissue to effect electrosurgical cutting or traversal. The depicted electrically uninsulated exposed distal tip allows for cutting (perforating and traversing) tissue. In addition, the mid-shaft of the device allows cutting (lacerating) soft tissue as described herein. Implementations of the depicted guidewire preferably include a sterile, single use device intended to cut soft tissue. References to dimensions and other specific information in this Appendix is intended to be illustrative and non-limiting. In one implementation, the disclosed guidewire has an outer diameter of 0.014″ and a working length of 260-300 cm. The proximal end of the disclosed guidewire, which has no patient contact, can be un-insulated to allow for connection to an electrosurgery generator. The electrosurgery generator can be the Medtronic Force FX C Generator that achieves 20 W to 100 Watts of monopolar radiofrequency (RF) energy, for example.

If desired, the disclosed guidewire can include a mid-shaft insulator to protect the operator from electrosurgical energy when the guidewire tip is used for electrosurgical traversal cutting inside the patient. The disclosed guidewire can be accompanied by a detachable spring-loaded connector cable that plugs into the Medtronic Force FX C generator and allows for a secure insulative connection to the generator. The detachable connector allows for fast and easy exchange of catheters over the disclosed guidewire. Two additional accessories can be provided; a wire gripper and a kinker block. The wire gripper can resemble a standard guidewire torquer to assist with guidewire traction when using the mid-shaft surface for electrosurgical cutting. The kinker block can be provided to create a reproducible kink aligned with the un-insulated portion to create a focused cutting surface for laceration. Thus, the illustrated embodiment includes a guidewire, a spring-loaded connector cable, a wire gripper, and a kinker block. FIG. 6 depicts the depicted guidewire with associated cross-sectional views. FIGS. 7-9 depict images of additional accessories for the disclosed guidewire. A spring-loaded connector cable as depicted in FIG. 7, a wire gripper (FIG. 8) and a kinker block (FIG. 9) are depicted. There are two cutting surfaces of the illustrated guidewire of FIG. 6, such as the distal tip and a mid-shaft cutting location approximately 150 cm from the distal tip. The mid-shaft cutting location can be about 5 mm of locally un-insulated stainless-steel guidewire and it is not introduced into the patient unless it is required during the procedure. The mid-shaft cutting surface can be covered by a removable insulator when not in use. The guidewire can be used in conjunction with a guide catheter to access tissue such as a valve leaflet and the distal tip is electrified to puncture through the tissue. The distal tip of the guidewire can be captured by a snare as described above and externalized. The central un-insulated portion of the wire can be intentionally kinked, and advanced to the desired location on the tissue and RF energy can be applied to cut through tissue while exerting traction on the wire.

The guidewire can be composed of a 304V stainless steel guidewire covered with an outer insulative layer. The distal tip, the mid-shaft cutting surface (center section of wire) and proximal end can be denuded of insulation. The mid-shaft cutting surface preferably does not contact the patient during electrosurgery using the distal tip. The distal tip typically does not contact the patient during electrosurgery using the mid-shaft. The proximal end typically does not contact the patient. The generator connector cable, wire gripper, and kink blocker are preferably non-patient contact parts and are constructed of standard materials.

The guidewire of FIG. 6 can be placed through a standard introducer sheath and guided under fluoroscopy to the site of use. The spring-loaded connector cable on the proximal portion of the guidewire can be connected to the RF generator (Force FX C) and the distal tip of the TELLTALE guidewire can be advanced through the tissue. The mid-shaft cutting surface can be intentionally kinked using the kinker block of FIG. 9 and introduced into the patient to allow for tissue cutting using monopolar RF energy. The mid-shaft cutting surface of the TELLTALE guidewire can be temporarily shielded and not introduced into the patient until it is needed during the procedure.

A common application of the guidewire can be BASILICA (Bioprosthetic Aortic Scallop Intentional Laceration to prevent Iatrogenic Coronary Artery obstruction during transcatheter aortic valve replacement). The procedure is performed under general anesthesia or under moderate sedation at the discretion of the institutional heart team. The BASILICA procedure typically has three steps as described elsewhere in this patent application, including (i) leaflet traversal by cutting using the distal guidewire tip, followed by (ii) leaflet laceration by cutting using the guidewire mid-shaft lacerating surface, immediately followed by (iii) TAVR using devices marketed outside the scope of this IDE. These steps are all typically guided by fluoroscopy and adjunctive echocardiography as needed.

First, catheter access is obtained typically via multiple arterial introducer sheaths. At various steps of the procedure, two or four catheters can be used for BASILICA (often with catheter pairs introduced side-by-side into single large-bore introducer sheaths), one for hemodynamics and angiography, and one for TAVR) and at least one venous introducer sheath for temporary transvenous pacing. Anticoagulation with heparin or equivalent achieves an activated clotting time is typically 250-300s. Cerebral embolic protection devices are employed at the discretion of the operator. Two retrograde catheters are positioned, using a guidewire anchor as needed, in the LVOT and Aorta respectively. Care is taken to avoid entrapment of mitral valvular structures. A snare catheter is positioned in the LVOT. A traversal guiding catheter directs the TELLTALE guidewire against the base of the coronary cusp targeted for laceration, using fluoroscopic and/or echocardiographic guidance.

Traversal cutting is accomplished by transcatheter electrosurgery by connecting the electrically exposed proximal end of the guidewire to a spring-loaded connector cable to facilitate short bursts of “pure, cutting” radiofrequency energy typically at approximately 20 W-50 W. The guidewire is repositioned as needed until it crosses the aortic leaflet and is snare-retrieved and externalized as described above.

Next, the temporary shielding over the exposed region of the middle of the guidewire is removed and the center denuded section of the guidewire is intentionally kinked using a kinker block (e.g., FIG. 9) to enforce its position at the inner curvature of the intended guidewire lacerating surface. The ensnared guidewire is externalized to position the lacerating surface across the base of the leaflet. The kink self-orients the denuded lacerating surface with the leaflet tissue intended to be cut. Nonionic conductive flush (e.g., dextrose 5%) is administered as needed during electrosurgery via the guiding catheters to reduce non-target electrical pathways and to reduce guidewire char and thromboembolism. The BASILICA procedure may be performed on one or two valve leaflets that may threaten coronary artery obstruction.

Laceration cutting can be performed by positioning the laceration (denuded mid-shaft) surface along the intended leaflet base, and applying traction on both free ends of the guidewire with the wire grippers while simultaneously apply electrosurgery energy (typically 50-70 W) in short bursts, until the laceration is complete and the guidewire is free. The guidewire and BASILICA catheters are removed. With the leaflets cut, installation of a TAVR is then performed as usual.

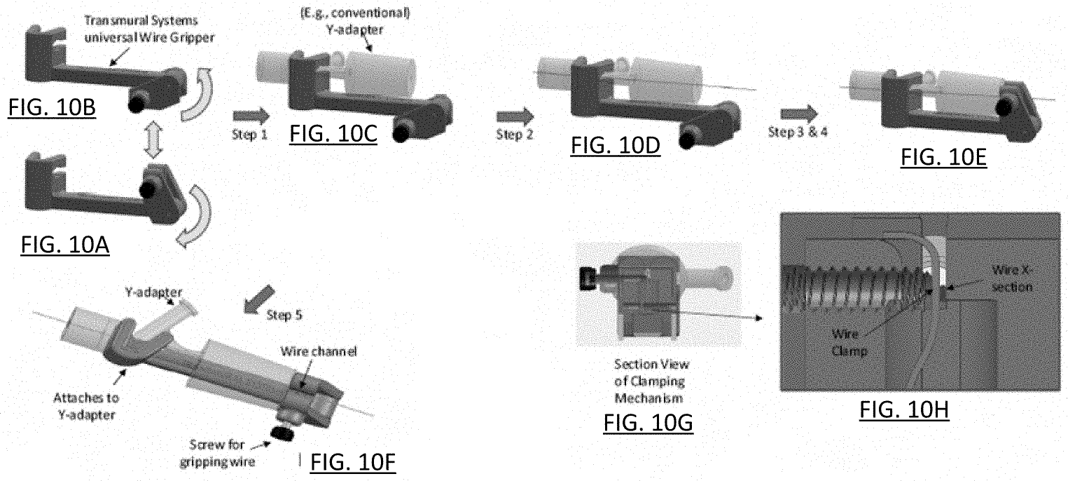

FIGS. 10A-12B depict a further implementation of a guidewire gripper that can be used with a Y-Adapter, such as a typical off the shelf Y-Adapter. The guidewire gripper includes an elongate frame or body, illustrated as a rod or shaft. A first end of the gripper includes at least one gripping arm to hold a neck portion of a Y-Adapter in place. The at least one gripping arm is set forth as a flange located at the first end of the gripper that extends laterally outwardly from the main shaft or frame of the gripper and defines a pair of gripping arms. A second end of the frame includes a wire clamp pivotally attached thereto that rotates about a hinge ping that passes through the second end of the frame. In use, the gripping arms of the gripper snap over a neck portion of a Y-adapter (Step 1, FIG. 10C). The guidewire is then inserted through the Y-adapter (Step 2, FIG. 10D). The wire clamp at the second (“proximal”) end of the frame is then rotated up (FIG. 10E) to cause the guidewire to fall into a channel of the clamp of the gripper as illustrated in the cross-sectional views (FIGS. 10G, 10H). The manual screw can then be advanced into the channel of the clamp to clamp the guidewire in place between a distal face of the screw and a further portion of the clamp, such as a grip plate.

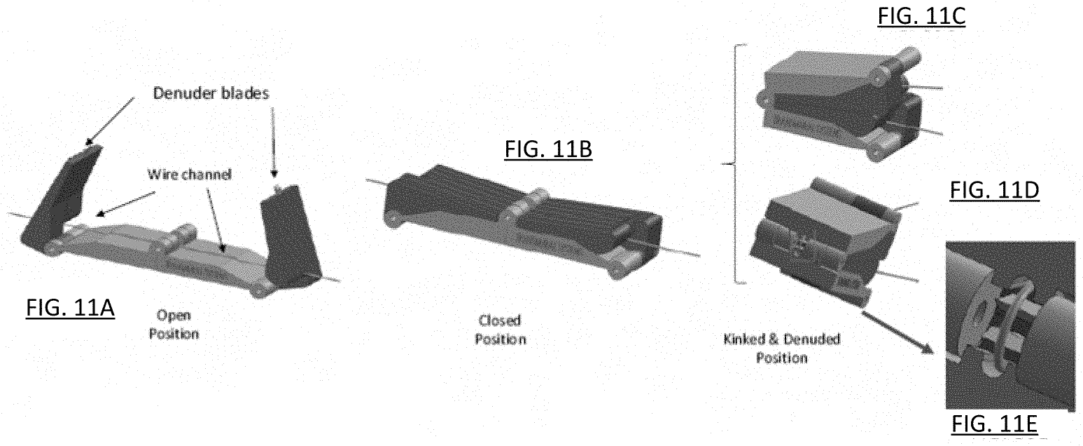

FIGS. 11A-11E illustrates a further kinker or denuder to remove a coating from a guidewire, typically a dielectric coating such as PTFE. The denuder includes two central frame portions joined at a central hinge that, when unfolded into an elongate configuration, defines an elongate wire channel along an upper side of the two central frame portions. Each central frame portion includes a further hinge at an outer end of the central frame portions that are connected by way of a hinge pin to a respective articulating arm that is connected to a respective central frame portion at a first end, and that includes a blade at a second free end thereof. As illustrated, the guidewire is introduced into the elongate channel defined along an upper face of the two central frame portions, the guidewire passing between two hinge bosses at a center of the kinker/denuder. The wire passes along a first lateral side of a first articulating arm, and a second opposing side of the second articulating arm as illustrated in the open position. The kinker/denuder is then closed first by collapsing each articulating arm toward its respective central frame portion. This places the kinker/denuder into the illustrated “closed position”. The kinker/denuder is then folded again about its central hinge to collapse it and to bend the guidewire over onto itself at an acute angle. During this folding process, the guidewire contacts the denuder blades and scrapes the coating from the inside surface of the guidewire and, if so configured, can add a bend, or a kink to the guidewire.

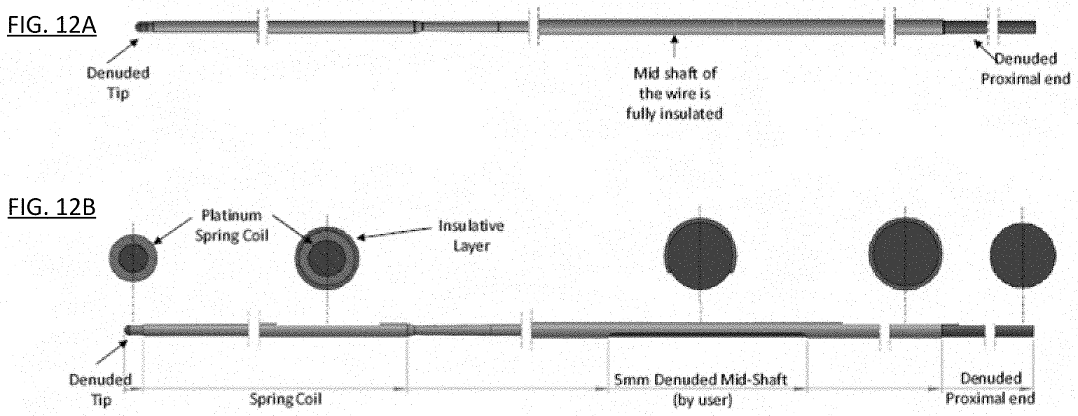

FIGS. 12A-12B includes two embodiments of a guidewire, similar to other embodiments herein that each include a denuded proximal end and a denuded distal end to permit the wire to burn through tissue by way of its distal tip when it is electrified. The distal end region of the wire includes a platinum coil placed over the distal seven centimeters of the core wire, and that in turn is coated with an electrically insulating layer, with the exception of the distal tip of the wire, such as the last 1.5 mm of the wire. If desired, the second variation shows a denuded region about 5 mm long that is about 150 cm from the distal tip to permit an electrified procedure to be performed at the location of the denuded region. The devices and methods of can be used to help effectuate the procedures set forth herein.

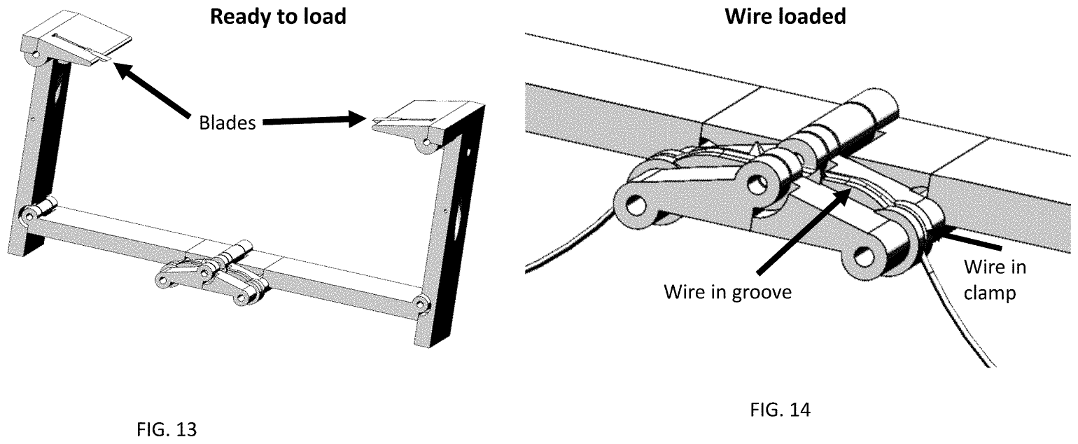

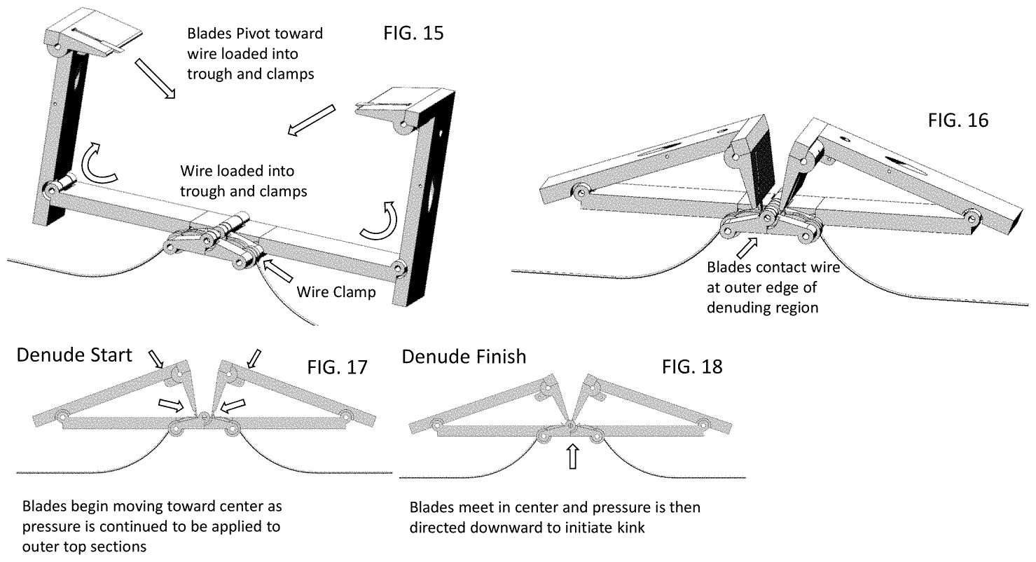

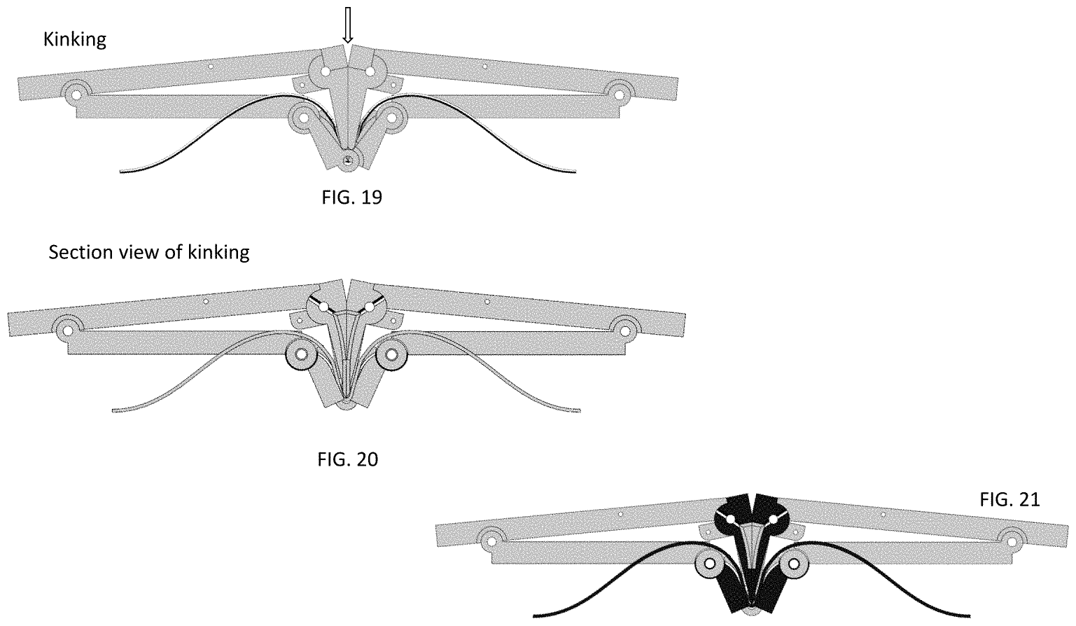

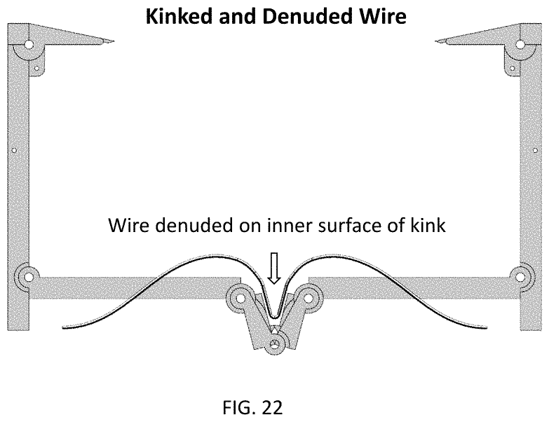

Still a further implementation of a kinker block is depicted in FIGS. 13-22. The kinker block includes a main horizontal body portion coupled at either end by a pin to an upright pivoting arm, wherein each pivoting arm terminates in a pivoting knuckle that in turn includes a blade. The wire is laid in a groove per FIG. 14 and placed in a clamp that is parallel to the horizontal body portion. Per FIG. 15, the blades pivot toward the wire loaded into the groove and clamps, wherein the blades contact the wire at an outer edge of the denuding region (FIG. 16). The blades begin moving toward the center as pressure is continued to be applied to the outer top sections (FIG. 17). The blades then meet in the center and pressure is then directed downward to initiate formation of a kink in the wire (FIG. 18). FIGS. 19-21 illustrate the kinking process and FIG. 22 illustrates the kinked denuded wire in the kinker after the operation is completed.

The devices and methods disclosed herein can be used for other procedures in an as-is condition, or can be modified as needed to suit the particular procedure. In view of the many possible embodiments to which the principles of this disclosure may be applied, it should be recognized that the illustrated embodiments are only preferred examples of the disclosure and should not be taken as limiting the scope of the disclosure.

What is claimed is:

1. An electrosurgical guidewire, comprising:
a core wire having a proximal end, and a distal end and being defined by an outer surface between the proximal end and the distal end of the core wire, said core wire having a centerline that traverses the length of the core wire from the proximal end to the distal end of the core wire;
radiopaque markers disposed over the core wire to indicate a location proximate a middle section of the guidewire to be kinked and used to cut through tissue during an electrosurgical procedure; and
a dielectric coating disposed about the core wire and the radiopaque marker pattern, wherein the proximal end and distal end of the core wire are exposed and the proximal end is configured to be coupled to an electrosurgical generator, and further wherein the dielectric coating is configured to be stripped from the guidewire proximate the radiopaque marker pattern,

2. The electrosurgical guidewire of claim 1, wherein the radiopaque marker pattern defines a central region to be crimped and stripped of the dielectric coating.

3. The electrosurgical guidewire of claim 1, further comprising a radiopaque coil surrounding the distal tip of the guidewire.

4. An elongate catheter having a proximal end and a distal end comprising:
a) an elongate tubular main body having a proximal end, a distal end, and defining at least one elongate passage therethrough, the elongate tubular main body defining a longitudinal axis along its length;
b) a first elongate inner body having a proximal end and a distal end that is slidably disposed within the at least one elongate passage of the elongate tubular main body, the distal end of the first inner elongate body being configured to curl away from the longitudinal axis in a proximal direction when the first elongate inner body is advanced distally with respect to the main body; and
c) a second elongate inner body having a proximal end and a distal end that is slidably disposed within the at least one elongate passage of the elongate tubular main body, and slidably disposed with respect to the first inner body, the distal end of the second inner elongate body being configured to curl away from the longitudinal axis toward the deployed proximally oriented distal end of the first elongate inner body when the second elongate inner body is advanced distally with respect to the main body, wherein:
each of the first elongate inner body and second elongate inner body each define a lumen along their respective lengths;
the lumen of the second elongate inner body includes a movable body slidably disposed therein having a distal end, the movable body including a guidewire having an electrically conductive core wire surrounded by a jacket made from dielectric material, wherein the jacket is removed from a portion of the core wire along one side of the core wire to create an exposed region of the core wire.

5. The elongate catheter of claim 4, wherein the lumen of the first elongate inner body includes a snare catheter configured to be deployed from the distal end of the first elongate inner body to capture the guidewire when the guidewire is deployed from the distal end of the second elongate inner body.

6. The elongate catheter of claim 4, wherein the core wire is surrounded by a first insulating layer, the first insulating layer is surrounded by a second electrical conductor, and the second electrical conductor is surrounded by an outer insulating layer, wherein the outer insulating layer is removed to expose a portion of the second electrical conductor to define an exposed portion of the second electrical conductor.

7. The elongate catheter of claim 6, wherein the second electrical conductor is formed from an electrically conductive tube.

8. The elongate catheter of claim 7, wherein the electrically conductive tube is defined by a tubular body that in turn defines at least one opening therethrough.

9. The elongate catheter of claim 7, wherein the at least one opening is spiral shaped and winds around the first insulating layer.

10. The elongate catheter of claim 7, wherein the at least one opening and the tubular body define a plurality of articulating segments.

11. An electrosurgical system including a radio frequency power supply operably coupled to first and second electrically conductive ends of the electrically conductive core wire of the elongate catheter of claim 4.

12. The elongate catheter of claim 4, wherein the core wire is bent in half so that the exposed region of the core wire faces itself.

13. A system including the elongate catheter of claim 12, wherein the core wire includes electrically conductive exposed first and second ends coupled to an electrosurgical generator to cause current to pass through the core wire to cause an arc discharge to develop in the exposed region of the core wire that jumps across a gap between facing exposed surfaces of the core wire in the exposed region of the core wire that can help cut and/or burn through tissue by pulling the exposed wire through the tissue.
